(19) United States
(12) Patent Application Publication
(10) Pub. No.: US 2014/0163061 A1
(43) Pub. Date: Jun. 12, 2014

(54) Methods For Reducing Blood Pressure

(71) Applicants: Volkmar Guenzler-Pukall, Marburg (DE); Qingjian Wang, Belmont, CA (US); Ingrid Langsetmo Parobok, Fremont, CA (US); Guangjie Guo, Foster City, CA (US)

(72) Inventors: Volkmar Guenzler-Pukall, Marburg (DE); Qingjian Wang, Belmont, CA (US); Ingrid Langsetmo Parobok, Fremont, CA (US); Guangjie Guo, Foster City, CA (US)

(73) Assignee: FibroGen, Inc., San Francisco, CA (US)

(21) Appl. No.: 14/180,720

(22) Filed: Feb. 14, 2014

Related U.S. Application Data
(63) Continuation of application No. 12/734,444, filed on Oct. 25, 2010, now abandoned.

(51) Int. Cl. C07D 471/04; C07D 217/26

(52) U.S. Cl. CPC C07D 471/04; C07D 217/26
USPC 514/300; 514/307

(57) ABSTRACT

The present invention provides methods and medicaments for reducing blood pressure. Methods and medicaments for treating or preventing hypertension are also provided.

This application is a continuation of U.S. application Ser. No. 12/734,444, filed 25 Oct. 2010 pursuant to 35 U.S.C. §371 as a United States National Phase Application of International Patent Application No. PCT/US2008/012449, filed 3 Nov. 2008; which claims priority from Provisional Application No. 60/984,726 filed 2 Nov. 2007, each of which is incorporated by reference herein in its entirety.

### FIELD OF THE INVENTION

The present invention relates to methods and medicaments for reducing blood pressure and treating or preventing hypertension. In particular, methods and medicaments for treatment of hypertension and prehypertension and for reducing blood pressure in kidney disease patients are specifically provided herein. Specific compounds for use in methods and medicaments for treatment of hypertension and prehypertension and for reducing blood pressure in a subject, the subject including but not limited to a subject having kidney disease, are further provided.

### BACKGROUND OF THE INVENTION

Elevated blood pressure and hypertension are significant public health problems. Numerous risk factors are associated with elevations in blood pressure or the development of hypertension, including age, race, family history, obesity, inactivity, tobacco use, alcohol use, diet, diabetes, and stress. Generally, a subject may be considered prehypertensive upon consecutive readings at two or more occasions with a systolic pressure of from 120 to 139 mmHg or a diastolic pressure of from 80 to 89 mm Hg. A subject may be considered hypertensive upon consecutive readings at two or more occasions with systolic/diastolic pressure greater than or equal to 140/90 mmHg.

Individuals having elevated blood pressure or hypertension are at a significantly greater risk for developing numerous disorders and complications. The extent and severity of these disorders and complications suggest an urgent need for early and effective treatment strategies that reduce blood pressure and that treat hypertension or prevent/reverse the progression of hypertension. Relatively minor reductions in blood pressure can significantly reduce the co-morbidities and co-mortalities associated with hypertension. For example, in adults aged 40-69, a 20 mmHg reduction in systolic blood pressure (approximately equivalent to a 10 mmHg reduction in diastolic blood pressure) was associated with a greater than two-fold reduction in death due to stroke and other vascular diseases. (Lewington et al. (2002) Lancet 360:1903-1913.)

Individuals with elevated blood pressure, including hypertensive and prehypertensive subjects, are a heterogeneous population. This is due, in part, to the multifactorial etiology and numerous underlying mechanisms associated with elevated blood pressure. (Welsh et al. (2004) Int J Clin Pract. 58.956-63.) For example, elevated blood pressure may be caused by other underlying diseases such as chronic kidney disease or heart failure. The heterogeneity of these patient populations results in a varied response to antihypertensive therapy. (Laragh et al. Hypertension 12:223-226.)

The most frequently used blood pressure medications include: diuretics; β-blockers (BBs); angiotensin-converting enzyme (ACE) inhibitors; angiotensin receptor blockers (ARBs); and calcium channel blockers (CCBs). However, each of these major classes of antihypertensive drugs has both advantages and disadvantages that vary with underlying diseases that may be present. (Brunner et al. (1990) J Hypertens. 8:3-11.) For example, diuretics are often contraindicated in patients with kidney disease since volume depletion can result in reduction of blood flow to the kidneys and further deterioration of kidney function.

In addition, single drug-antihypertensive therapy is unsuccessful in up to two-thirds of all patients with hypertension. (Chobanian et al. (2003) Hypertension 42:1206-52.) Moreover, there is evidence of considerable variation in the response of different hypertensive patients to different drug classes. A number of treatment algorithms have been proposed in the literature, with a view to predicting an individual's response to different antihypertensive agents based on the presence or absence of a compelling indication (e.g., heart failure, chronic kidney disease, recurrent stroke prevention). (Chobanian et al. (2003) Hypertension 42:1206-52.) However, even using such algorithms, hypertension control remains problematic.

Therefore, there is a need in the art for methods and medicaments effective for reducing blood pressure and for treating hypertension and prehypertension. The present invention meets these needs by providing novel methods and medicaments for use in reducing blood pressure and in treating or preventing hypertension or prehypertension in subjects, including subjects having kidney disease. Such methods and medicaments can be used alone or in combination with current therapies to reduce blood pressure and treat hypertension or prehypertension in subjects in need thereof.

### SUMMARY OF THE INVENTION

The present invention relates generally to methods and medicaments for reducing blood pressure or preventing an increase in blood pressure and for treating or preventing hypertension or prehypertension in a subject having kidney disease. The invention further provides specific compounds effective in reducing blood pressure or preventing an increase in blood pressure and for treating or preventing hypertension or prehypertension in any subject, including, but not limited to, subjects having kidney disease.

The present invention provides methods and medicaments for treating or preventing hypertension or prehypertension in a subject having kidney disease. In one aspect, the invention provides methods for treating hypertension or prehypertension in a subject having kidney disease, the method comprising administering to the subject an effective amount of a compound that inhibits the activity of a hypoxia-inducible factor (HIF) prolyl hydroxylase enzyme, thereby treating hypertension or prehypertension in the subject. In another aspect, the invention provides methods for preventing hypertension in a subject having kidney disease, the method comprising administering to the subject an effective amount of a compound that inhibits the activity of a hypoxia-inducible factor (HIF) prolyl hydroxylase enzyme, thereby preventing hypertension in the subject having kidney disease. In a one aspect, the subject having kidney disease is prehypertensive. In another aspect, the invention provides for the use of a compound that inhibits the activity of a HIF prolyl hydroxylase enzyme in the manufacture of a medicament for treating or preventing hypertension or prehypertension in a subject having kidney disease.

In one embodiment, the present invention provides a method for reducing blood pressure or preventing an increase in blood pressure in a subject having kidney disease, the method comprising administering to the subject an effective amount of a compound that inhibits the activity of a hypoxia-inducible factor (HIF) prolyl hydroxylase enzyme, thereby reducing blood pressure or preventing an increase in blood pressure in the subject. Use of a compound that inhibits the activity of a HIF prolyl hydroxylase enzyme in the manufacture of a medicament for reducing blood pressure in a subject having kidney disease is also contemplated herein. In various embodiments of the present invention, methods for reducing systolic blood pressure, or for reducing diastolic blood pressure, or for reducing mean arterial pressure in a subject having kidney disease, the methods comprising administering to the subject an effective amount of a compound that inhibits the activity of a hypoxia-inducible factor (HIF) prolyl hydroxylase enzyme, thereby reducing systolic blood pressure, or reducing diastolic pressure, or reducing mean arterial pressure, respectively, are also contemplated.

In various embodiments of the present invention, the subject having kidney disease, or the kidney disease subject, is a subject having a disorder or disease of the kidney including, but not limited to, acute kidney failure, chronic kidney disease, end-stage renal disease, kidney damage, membranous nephropathy, and the like.

The subject having kidney disease can also be a subject at risk for high blood pressure or hypertension due to a disorder selected from the group consisting of chronic kidney disease, acute kidney failure, and renal insufficiency. Such subjects can include a subject having one or more of various factors known to be associated with an increased risk of developing elevated or high blood pressure or hypertension. Such risk factors include, for example, family history of high blood pressure, diabetes, obesity, certain ethnicity or race, a sedentary lifestyle, age, alcohol use, tobacco use, caffeine use, diet, sodium sensitivity and salt intake, kidney disease and renal insufficiency, sleep apnea, pregnancy, cirrhosis, Cushing's disease, certain medications, emotional factors, stress, etc.

The compounds for use in the methods and medicaments for treating or preventing hypertension or prehypertension, or reducing blood pressure, in a subject having kidney disease are inhibitors of HIF prolyl hydroxylase enzymes. In particular embodiments, the compounds used in the methods and medicaments for treating hypertension in a subject having kidney disease are structural mimetics of 2-oxoglutarate, which may inhibit the target HIF prolyl hydroxylase enzyme competitively with respect to 2-oxoglutarate and noncompetitively with respect to iron. In another embodiment, compounds for use in the present methods and medicaments are heterocyclic carbonyl glycines of formula A:

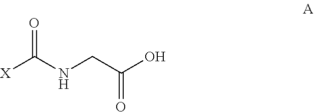

wherein X is an optionally substituted heterocyclic moiety.

In specific embodiments, such compounds include, but are not limited to, substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, and 4-hydroxy-pyrrolopyridine-5-carbonyl-glycines. Compounds can be identified for use in the present embodiments by measuring inhibitory activity of the compound on a HIF prolyl hydroxylase enzyme, e.g., using an enzyme assay as described herein. More generally, compounds can be identified for use in the present embodiments by measuring HIF stabilization induced by the compound, e.g., using a cell-based assay as described herein. (See, e.g., Examples 1 and 2.)

In addition to the methods and medicaments described above, the present invention provides for use of compounds of formula I in methods for treating or preventing hypertension or prehypertension, or reducing blood pressure, in a subject, the method comprising administering to the subject an effective amount of a compound of formula I:

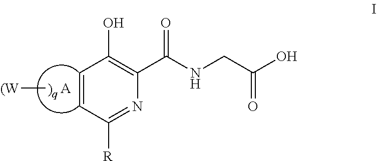

wherein
A is selected from a benzene or pyrrole ring;
q is 1, 2 or 3;
W is selected from (C₁-C₃)-alkyl, (C₁-C₃)-alkoxy or (C₆-C₁₀)-aryloxy, each of which may be unsubstituted or substituted by one or more halo, (C₁-C₃)-alkyl, (C₁-C₃)-alkoxy, or (C₆-C₁₀)-aryl; and
R is selected from hydrogen, alkyl, or cyano;
or a pharmaceutically acceptable salt, ester, or prodrug thereof.

Use of a compound of formula I to manufacture a medicament for the treatment of hypertension or prehypertension in a subject is also provided herein.

In one embodiment, the present invention provides a method for reducing blood pressure or preventing an increase in blood pressure in a subject in need thereof, the method comprising administering to the subject an effective amount of a compound of formula I, thereby reducing blood pressure or preventing an increase in blood pressure in the subject. Use of a compound of formula I in the manufacture of a medicament for reducing blood pressure or preventing an increase in blood pressure in a subject in need thereof is also contemplated herein. In various embodiments of the present invention, methods for reducing systolic blood pressure, or for reducing diastolic blood pressure, or for reducing mean arterial pressure in a subject in need, the methods comprising administering to the subject an effective amount of a compound of formula I, thereby reducing systolic blood pressure, or reducing diastolic pressure, or reducing mean arterial pressure in the subject, respectively, are also contemplated.

In one embodiment, the present invention provides methods and medicaments for preventing hypertension in a subject having prehypertension, the method comprising administering to the subject an effective amount of a compound of formula I, thereby preventing hypertension in the subject having prehypertension. In certain embodiments of the present aspect, the subject is at risk for developing elevated or high blood pressure, hypertension, or prehypertension. Such subjects can include a subject having one or more of various factors known to be associated with an increased risk of developing elevated or high blood pressure or hypertension. Such risk factors include, for example, family history of high blood pressure, diabetes, obesity, certain ethnicity or race, a sedentary lifestyle, age, alcohol use, tobacco use, caffeine use, diet, sodium sensitivity and salt intake, kidney disease and renal insufficiency, sleep apnea, pregnancy, cirrhosis, Cushing's disease, certain medications, emotional factors, stress, etc. In one embodiment, the subject at risk of high blood pressure or hypertension has a disorder selected from the group consisting of kidney disease and renal insufficiency. In various embodiments, the subject at risk of high blood pressure or hypertension is non-anemic. In some embodiments, the high blood pressure or hypertension is associated with kidney disease.

In certain embodiments of the present invention, a compound of formula I is administered to a subject to treat hypertension, wherein the hypertension is further selected from the group consisting of mild hypertension, moderate hypertension, severe hypertension, and very severe hypertension.

In various embodiments of the present invention, the subject is a mammalian subject. In particular embodiments, the subject is a human subject.

In some embodiments, the subject had been previously treated with or is currently being treated with one or more blood pressure medications including, but not limited to, ACE inhibitors (e.g., benazepril, fosinopril, lisinopril, quinapril), ARBs (e.g., losartan), BBs (e.g., metoprolol tartrate, betaxolol, valsartan), diuretics (e.g., hydrochlorothiazide), vasodilators (e.g., isosorbide dinitrate), α-blockers, CCBs, and statins. In particular embodiments, the methods of the present invention further comprise treatment of the subject with a second therapeutic compound selected from the group comprising ACE inhibitors, ARBs, a-blockers, BBs, vasodilators, CCBs, and statins.

In some embodiments of the present aspect, the compound is selected from a compound of formula I(a):

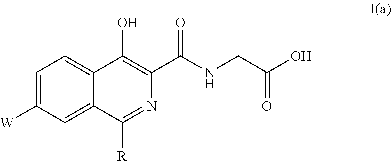

wherein W and R are as defined above, or a pharmaceutically acceptable salt, ester, or prodrug thereof.

In particular embodiments, the compound of formula I(a) is selected from compounds wherein W is selected from (C₁-C₃)-alkoxy or (C₆-C₁₀)-aryloxy, each of which may be unsubstituted or substituted by one or more (C₁-C₃)-alkyl and/or (C₁-C₃)-alkoxy; and R is selected from hydrogen, alkyl, or cyano; or a pharmaceutically acceptable salt, ester, or prodrug thereof.

In other embodiments of the present aspect, the compound is selected from a compound of formula I(b):

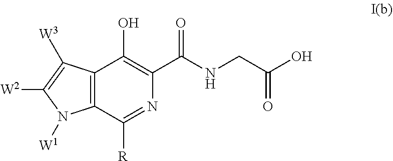

wherein
W¹ is selected from (C₁-C₃)-alkyl, which may be unsubstituted or substituted by one or more (C₁-C₃)-alkoxy or (C₆-C₁₀)-aryl;
W² and W³ are each independently selected from halo or (C₁-C₃)-alkyl; and
R is selected from hydrogen, alkyl, or cyano;
or a pharmaceutically acceptable salt, ester, or prodrug thereof.

In various embodiments of the present invention, the compound for use in the methods or for use in manufacture of a medicament is selected from the group consisting of {[4-hydroxy-7-(4-methoxy-phenoxy)-isoquinoline-3-carbonyl]-amino}-acetic acid, [(4-hydroxy-1-methyl-7-phenoxy-isoquinoline-3-carbonyl)-amino]-acetic acid, [(1-cyano-4-hydroxy-5-phenoxy-isoquinoline-3-carbonyl)-amino]-acetic acid, [(1-cyano-4-hydroxy-7-isopropoxy-isoquinoline-3-carbonyl)-amino]-acetic acid, {[1-cyano-7-(2,6-dimethyl-phenoxy)-4-hydroxy-isoquinoline-3-carbonyl]-amino}-acetic acid, {[3-bromo-7-cyano-4-hydroxy-1-(4-methoxy-benzyl)-1H-pyrrolo[2,3-c]pyridine-5-carbonyl]-amino}-acetic acid, {[3-chloro-7-cyano-4-hydroxy-1-(4-methoxy-benzyl)-1H-pyrrolo[2,3-c]pyridine-5-carbonyl]-amino}-acetic acid, {[2,3-dichloro-7-cyano-4-hydroxy-1-(4-methoxy-benzyl)-1H-pyrrolo[2,3-c]pyridine-5-carbonyl]-amino}-acetic acid, {[7-cyano-1-(2-fluoro-benzyl)-4-hydroxy-1H-pyrrolo[2,3-c]pyridine-5-carbonyl]-amino}-acetic acid, {[7-cyano-4-hydroxy-1-(3-methoxy-benzyl)-1H-pyrrolo[2,3-c]pyridine-5-carbonyl]-amino}-acetic acid, [(7-cyano-4-hydroxy-1-naphthalen-2-ylmethyl-1H-pyrrolo[2,3-c]pyridine-5-carbonyl)-amino]-acetic acid.

In certain embodiments of the present invention, a compound of formula I is administered to a subject to treat hypertension, wherein the hypertension is further selected from the group consisting of mild hypertension, moderate hypertension, severe hypertension, and very severe hypertension.

In various embodiments of the present invention, the subject is a mammalian subject. In particular embodiments, the subject is a human subject.

In some embodiments, the subject had been previously treated with or is currently being treated with one or more blood pressure medications including, but not limited to, ACE inhibitors (e.g., benazepril, fosinopril, lisinopril, quinapril), ARBs (e.g., losartan), BBs (e.g., metoprolol tartrate, betaxolol, valsartan), diuretics (e.g., hydrochlorothiazide), vasodilators (e.g., isosorbide dinitrate), α-blockers, CCBs, and statins. In particular embodiments, the methods of the present invention further comprise treatment of the subject with a second therapeutic compound selected from the group comprising ACE inhibitors, ARBs, α-blockers, BBs, vasodilators, diuretics, CCBs, and statins.

These and other aspects and embodiments of the present invention will readily occur to those of skill in the art in light of the disclosure herein, and all such aspects and embodiments are specifically contemplated.

### BRIEF DESCRIPTION OF THE DRAWINGS

FIG. 1 sets forth data showing that methods of the present invention effectively reduced systolic blood pressure in mammalian subjects.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a compound” may include a plurality of such compounds and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

### DETAILED DESCRIPTION

The present invention relates generally to methods and medicaments for reducing blood pressure or preventing an increase in blood pressure and for treating or preventing hypertension or prehypertension in a subject having kidney disease. The invention further provides specific compounds effective in reducing blood pressure or preventing an increase in blood pressure and for treating or preventing hypertension or prehypertension in any subject, including, but not limited to, subjects having kidney disease.

The present invention provides methods and medicaments for treating or preventing hypertension or prehypertension in a subject having kidney disease. In one aspect, the invention provides methods for treating hypertension or prehypertension in a subject having kidney disease, the method comprising administering to the subject an effective amount of a compound that inhibits the activity of a hypoxia-inducible factor (HIF) prolyl hydroxylase enzyme, thereby treating hypertension or prehypertension in the subject. In another aspect, the invention provides methods for preventing hypertension in a subject having kidney disease, the method comprising administering to the subject an effective amount of a compound that inhibits the activity of a hypoxia-inducible factor (HIF) prolyl hydroxylase enzyme, thereby preventing hypertension in the subject having kidney disease. In a one aspect, the subject having kidney disease is prehypertensive. In another aspect, the invention provides for the use of a compound that inhibits the activity of a HIF prolyl hydroxylase enzyme in the manufacture of a medicament for treating or preventing hypertension or prehypertension in a subject having kidney disease.

In one embodiment, the present invention provides a method for reducing blood pressure or preventing an increase in blood pressure in a subject having kidney disease, the method comprising administering to the subject an effective amount of a compound that inhibits the activity of a hypoxia-inducible factor (HIF) prolyl hydroxylase enzyme, thereby reducing blood pressure or preventing an increase in blood pressure in the subject. Use of a compound that inhibits the activity of a HIF prolyl hydroxylase enzyme in the manufacture of a medicament for reducing blood pressure in a subject having kidney disease is also contemplated herein. In various embodiments of the present invention, methods for reducing systolic blood pressure, or for reducing diastolic blood pressure, or for reducing mean arterial pressure in a subject having kidney disease, the methods comprising administering to the subject an effective amount of a compound that inhibits the activity of a hypoxia-inducible factor (HIF) prolyl hydroxylase enzyme, thereby reducing systolic blood pressure, or reducing diastolic pressure, or reducing mean arterial pressure, respectively, are also contemplated.

In various embodiments of the present invention, the subject having kidney disease, or the kidney disease subject, is a subject having a disorder or disease of the kidney including, but not limited to, acute kidney failure, chronic kidney disease, end-stage renal disease, kidney damage, membranous nephropathy, and the like.

The subject having kidney disease can also be a subject at risk for high blood pressure or hypertension due to a disorder selected from the group consisting of chronic kidney disease, acute kidney failure, and renal insufficiency. Such subjects can include a subject having one or more of various factors known to be associated with an increased risk of developing elevated or high blood pressure or hypertension. Such risk factors include, for example, family history of high blood pressure, diabetes, obesity, certain ethnicity or race, a sedentary lifestyle, age, alcohol use, tobacco use, caffeine use, diet, sodium sensitivity and salt intake, kidney disease and renal insufficiency, sleep apnea, pregnancy, cirrhosis, Cushing's disease, certain medications, emotional factors, stress, etc.

In addition to the methods and medicaments described above, the present invention provides for use of compounds of formula I in methods for treating or preventing hypertension or prehypertension, or reducing blood pressure, in a subject, the method comprising administering to the subject an effective amount of a compound of formula I:

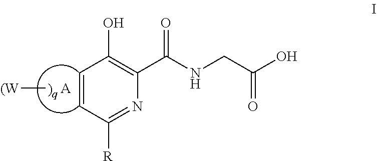

wherein
A is selected from a benzene or pyrrole ring;
q is 1, 2 or 3;
W is selected from (C₁-C₃)-alkyl, (C₁-C₃)-alkoxy or (C₆-C₁₀)-aryloxy, each of which may be unsubstituted or substituted by one or more halo, (C₁-C₃)-alkyl, (C₁-C₃)-alkoxy, or (C₆-C₁₀)-aryl; and
R is selected from hydrogen, alkyl, or cyano;
or a pharmaceutically acceptable salt, ester, or prodrug thereof.

Use of a compound of formula I to manufacture a medicament for the treatment of hypertension or prehypertension in a subject is also provided herein.

In one embodiment, the present invention provides a method for reducing blood pressure or preventing an increase in blood pressure in a subject in need thereof, the method comprising administering to the subject an effective amount of a compound of formula I, thereby reducing blood pressure or preventing an increase in blood pressure in the subject. Use of a compound of formula I in the manufacture of a medicament for reducing blood pressure or preventing an increase in blood pressure in a subject in need thereof is also contemplated herein. In various embodiments of the present invention, methods for reducing systolic blood pressure, or for reducing diastolic blood pressure, or for reducing mean arterial pressure in a subject in need, the methods comprising administering to the subject an effective amount of a compound of formula I, thereby reducing systolic blood pressure, or reducing diastolic pressure, or reducing mean arterial pressure in the subject, respectively, are also contemplated.

In one embodiment, the present invention provides methods and medicaments for preventing hypertension in a subject having prehypertension, the method comprising administering to the subject an effective amount of a compound of formula I, thereby preventing hypertension in the subject having prehypertension. In certain embodiments of the present aspect, the subject is at risk for developing elevated or high blood pressure, hypertension, or prehypertension. Such subjects can include a subject having one or more of various factors known to be associated with an increased risk of developing elevated or high blood pressure or hypertension. Such risk factors include, for example, family history of high blood pressure, diabetes, obesity, certain ethnicity or race, a sedentary lifestyle, age, alcohol use, tobacco use, caffeine use, diet, sodium sensitivity and salt intake, kidney disease and renal insufficiency, sleep apnea, pregnancy, cirrhosis, Cushing's disease, certain medications, emotional factors, stress, etc. In one embodiment, the subject at risk of high blood pressure or hypertension has a disorder selected from the group consisting of kidney disease and renal insufficiency. In various embodiments, the subject at risk of high blood pressure or hypertension is non-anemic. In some embodiments, the high blood pressure or hypertension is associated with kidney disease.

In certain embodiments of the present invention, a compound of formula I is administered to a subject to treat hypertension, wherein the hypertension is further selected from the group consisting of mild hypertension, moderate hypertension, severe hypertension, and very severe hypertension.

In various embodiments of the present invention, the subject is a mammalian subject. In particular embodiments, the subject is a human subject.

The determination as to whether a subject has high blood pressure or hypertension can be made by any measure accepted and utilized by those skilled in the art. In a human subject, a systolic blood pressure below 120 mmHg and a diastolic blood pressure below 80 mmHg are generally considered normal or optimal. A systolic blood pressure of above 120 mmHg, but below 140 mmHg, or a diastolic blood pressure of above 80 mmHg but below 90 mmHg, may be considered prehypertensive. A systolic blood pressure of 140 mmHg or above, or a diastolic pressure of 90 mmHg or above, may be considered hypertensive. Significant health risks can occur in subjects having high blood pressure, particularly when the high blood pressure occurs in the presence of a condition such as diabetes mellitus, obesity, heart disease, kidney disease, smoking, or other associated risk factors. Thus, the present invention contemplates treatment of subjects having high normal blood pressure to prevent high blood pressure or hypertension. In certain embodiments, a human subject suitable for treatment using the present methods and medicaments is a subject having high blood pressure, particularly when the subject has a condition as described above.

The mean arterial pressure (MAP) represents a notional average blood pressure in a subject. MAP is defined as the average arterial pressure during a single cardiac cycle. Mean arterial pressure can be determined according to any method accepted and utilized by those skilled in the art. For example, mean arterial pressure can be calculated according to the following equation: (diastolic pressure+1/3 [systolic pressure−diastolic pressure]). (See Rogers et al. (2001) Ann Intern Med. 134:1024-32.) In one embodiment, the present invention provides methods and medicaments useful for reducing mean arterial pressure in subjects having elevated or high blood pressure or hypertension.

A human subject having a systolic blood pressure of greater than about 140 mmHg or a diastolic blood pressure of greater than about 90 mmHg is considered to have hypertension. Hypertension may be further classified as mild hypertension (Stage 1, systolic blood pressure of between 140 to 159 mmHg; diastolic blood pressure of between 90 to 99 mmHg), moderate hypertension (Stage 2, systolic blood pressure of between 160 to 179 mmHg; diastolic blood pressure of between 100 to 109 mmHg), severe hypertension (Stage 3, systolic blood pressure of between 180 to 209 mmHg; diastolic blood pressure of between 110 to 19 mmHg), or very severe hypertension (Stage 4, systolic blood pressure of greater than 210 mmHg; diastolic blood pressure of greater than 120 mmHg). Thus, in certain embodiments, a human subject suitable for treatment using the present methods and medicaments is a subject having hypertension including mild hypertension, moderate hypertension, severe hypertension, and very severe hypertension.

Essential hypertension, also known as primary or idiopathic hypertension, accounts for approximately 90% of all hypertension cases. The causes of essential hypertension are unknown, but may be associated with various complications and abnormalities in major organs and body systems, including the heart, kidneys, blood vessels, nerves, and hormones. The present invention provides methods and medicaments for treating essential hypertension in a subject. In one embodiment, the method comprises administering a compound of formula I to a patient in need thereof, thereby treating hypertension in the subject.

In other embodiments, the methods and medicaments can be used to treat a subject at risk for developing high blood pressure or hypertension. A subject at risk can be identified, for example, by an assessment of one or more various factors known to be associated with an increased risk of developing elevated or high blood pressure or hypertension. Such risk factors include, for example, family history of high blood pressure, diabetes, obesity, certain ethnic or racial heritage, a sedentary lifestyle, age, alcohol use, tobacco use, caffeine use, diet, sodium sensitivity and salt intake, kidney disease and renal insufficiency, sleep apnea, pregnancy, cirrhosis, Cushing's disease, certain medications, emotional factors, stress, etc. For example, elevated blood pressure and hypertension are frequently associated with kidney disease and various nephropathies. Thus, in various embodiments of the present invention, the subject in need is a subject having a disorder selected from the group consisting of kidney disease, including renal insufficiency. It is specifically contemplated herein that, in particular aspects, the subject at risk can be a subject without elevated blood pressure, e.g., a subject having normal or even lower than normal blood pressure, e.g., systolic blood pressure at or below 120 mmHg or diastolic blood pressure at or below 80 mmHg. Such subjects, however, will have an underlying condition such as kidney disease that increases their likelihood of developing high blood pressure or hypertension.

Hypertension is a common condition in subjects with kidney disease. (Agarwal et al. (2005) Hypertension 46:514-520.) Methods and medicaments of the present invention reduced blood pressure in human subjects with kidney disease. (See, e.g., Example 2.) Specifically, methods and medicaments of the present invention reduced systolic, diastolic and mean arterial pressure in human subjects with kidney disease. Therefore, methods and medicaments of the present invention are useful for reducing blood pressure in a subject with kidney disease. Further, methods and medicaments of the present invention are useful for treating hypertension associated with kidney disease in a subject.

Subjects with chronic kidney disease display a progressive increase in blood pressure with time. This well recognized phenomenon often results in the development of high blood pressure or hypertension in these subjects. (See, e.g., Example 1 and Example 2.) Methods and medicaments of the present invention prevented the elevation in blood pressure typically observed in these subjects. Therefore, the present invention provides methods and medicaments useful for preventing hypertension associated with kidney disease in a subject. In particular, the present invention demonstrates that administration of (HIF) prolyl hydroxylase inhibitors prevents hypertension associated with kidney disease, and in particular, reduces blood pressure.

In certain aspects, the subject at risk is a subject previously treated with or currently taking one or more blood pressure medications including, e.g., ACE inhibitors (e.g., benazepril, fosinopril, lisinopril, quinapril), ARBs (e.g., losartan), BBs (e.g., metoprolol tartrate, betaxolol, valsartan), diuretics (e.g., hydrochlorothiazide), vasodilators (e.g., isosorbide dinitrate), a-blockers, CCBs, and statins.

### Compounds

Compounds for use in the methods or medicaments provided herein are inhibitors of HIF prolyl hydroxylase enzymes. The term “HIF prolyl hydroxylase,” as used herein, refers to any enzyme that is capable of hydroxylating a proline residue within an alpha subunit of HIF. Such HIF prolyl hydroxylases include protein members of the EGL-9 (EGLN) 2-oxoglutarate- and iron-dependent dioxygenase family described by Taylor (2001) Gene 275:125-132; and characterized by Aravind and Koonin (2001) Genome Biol 2:RESEARCH0007; Epstein et al. (2001) Cell 107:43-54; and Bruick and McKnight (2001) Science 294:1337-1340. Examples of HIF prolyl hydroxylases include human SM-20 (EGLN1) (GenBank Accession No. AAG33965; Dupuy et al. (2000) Genomics 69:348-54), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511; Taylor, supra); mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF prolyl hydroxylase may include Caenorhabditis elegans EGL-9 (GenBank Accession No. AAD56365) and Drosophila melanogaster CGI114 gene product (GenBank Accession No. AAF52050). The term “HIF prolyl hydroxylase” also includes any active fragment of the foregoing full-length proteins.

A compound that inhibits the activity of a HIF prolyl hydroxylase enzyme is any compound that reduces or otherwise inhibits the activity of at least one HIF prolyl hydroxylase enzyme. Such compounds are referred to herein as “prolyl hydroxylase inhibitors” or “PHIs”. In particular embodiments, compounds used in the present methods and medicaments are structural mimetics of 2-oxoglutarate, wherein the compound inhibits the target HIF prolyl hydroxylase enzyme competitively with respect to 2-oxoglutarate and noncompetitively with respect to iron. Compounds that inhibit HIF prolyl hydroxylase enzyme activity have been described in, e.g., International Publication Nos. WO 03/049686, WO 02/074981, WO 03/080566, WO 2004/108681, WO 2006/094292, WO 2007/038571, WO 2007/090068, WO 2007/070359, WO 2007/103905, and WO 2007/115315.

In particular embodiments, the compounds used in the methods and medicaments for treating hypertension in a subject having kidney disease are structural mimetics of 2-oxoglutarate, which may inhibit the target HIF prolyl hydroxylase enzyme competitively with respect to 2-oxoglutarate and noncompetitively with respect to iron. In another embodiment, compounds for use in the present methods and medicaments are heterocyclic carbonyl glycines of formula A:

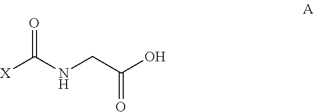

wherein X is an optionally substituted heterocyclic moiety. Such prolyl hydroyxlase inhibitors (PHIs) include, but are not limited to, variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, 4-hydroxy-pyrrolopyridine-5-carbonyl-glycines, etc.

In various embodiments of the present invention, the methods and medicaments provide for use of compounds of formula I:

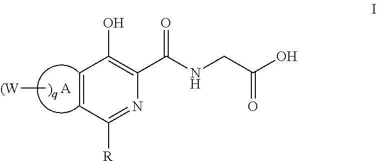

wherein
A is selected from a benzene or pyrrole ring;
q is 1, 2 or 3;
W is selected from (C₁-C₃)-alkyl, (C₁-C₃)-alkoxy or (C₆-C₁₀)-aryloxy, each of which may be unsubstituted or substituted by one or more halo, (C₁-C₃)-alkyl, (C₁-C₃)-alkoxy, or (C₆-C₁₀)-aryl; and
R is selected from hydrogen, alkyl, or cyano;
or a pharmaceutically acceptable salt, ester, or prodrug thereof.

In one embodiment, the method comprises administering to the subject an effective amount of a compound of formula I, thereby treating hypertension in the subject. In another embodiment, a compound of formula I is used in the manufacture of a medicament for reducing blood pressure in a subject in need thereof

The term “alkyl” refers to saturated monovalent hydrocarbyl groups and is exemplified by groups such as methyl, ethyl, n-propyl, iso-propyl, and the like. An alkyl substituted with one or more alkyl may include, but is not limited to, n-butyl, t-butyl, n-pentyl, 2-methyl-pentyl, 1-ethyl-2-methyl-pentyl, and the like. An alkyl substituted by an aryl may include, but is not limited to, benzyl, 1-naphthalen-2-yl-ethyl, and the like.

The term “alkoxy” refers to the group “alkyl-O—” and includes, by way of example, methoxy, ethoxy, n-propoxy, iso-propoxy, and the like.

The term “aryl” refers to a monovalent aromatic carbocyclic group having a single ring or multiple condensed rings and includes, by way of example, phenyl, naphthyl, and the like.

The term “aryloxy” refers to the group aryl-O— and includes, by way of example, phenoxy, naphthoxy, and the like.

The term “cyano” refers to the group —CN.

The term “halo” or “halogen” refers to fluoro, chloro, bromo, and iodo.

In some embodiments, the compound for use in the methods and medicaments is selected from a compound of formula I(a):

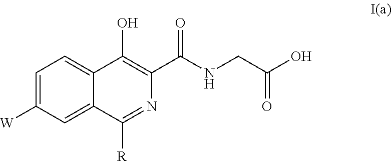

wherein W and R are as defined above, or a pharmaceutically acceptable salt, ester, or prodrug thereof.

In particular embodiments, the compound of formula I(a) is selected from compounds wherein W is selected from (C₁-C₃)-alkoxy or (C₆-C₁₀)-aryloxy, each of which may be unsubstituted or substituted by (C₁-C₃)-alkyl and/or (C₁-C₃)-alkoxy; and R is selected from hydrogen, alkyl, or cyano; or a pharmaceutically acceptable salt, ester, or prodrug thereof.

In other embodiments, the compound is selected from a compound of formula I(b):

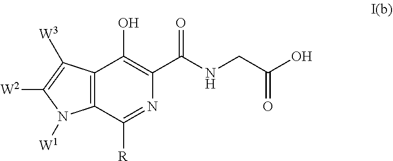

wherein
W¹ is selected from (C₁-C₃)-alkyl, which may be unsubstituted or substituted by one or more (C₁-C₃)-alkoxy or (C₆-C₁₀)-aryl;
W² and W³ are each independently selected from halo or (C₁-C₃)-alkyl; and
R is selected from hydrogen, alkyl, or cyano;
or a pharmaceutically acceptable salt, ester, or prodrug thereof.

In various embodiments of the present invention, the compound for use in the methods or for use in manufacture of a medicament is selected from the group consisting of [(4-hydroxy-1-methyl-7-phenoxy-isoquinoline-3-carbonyl)-amino]-acetic acid (A), {[4-hydroxy-7-(4-methoxy-phenoxy)-isoquinoline-3-carbonyl]-amino}-acetic acid (C), [(1-cyano-4-hydroxy-5-phenoxy-isoquinoline-3-carbonyl)-amino]-acetic acid (1), [(1-cyano-4-hydroxy-7-isopropoxy-isoquinoline-3-carbonyl)-amino]-acetic acid (D), {[1-cyano-7-(2,6-dimethyl-phenoxy)-4-hydroxy-isoquinoline-3-carbonyl]-amino}-acetic acid (J), {[3-bromo-7-cyano-4-hydroxy-1-(4-methoxy-benzyl)-1H-pyrrolo[2,3-c]pyridine-5-carbonyl]-amino}-acetic acid (K), {[2,3-dichloro-7-cyano-4-hydroxy-1-(4-methoxy-benzyl)-1H-pyrrolo[2,3-c]pyridine-5-carbonyl]-amino}-acetic acid (H), {[7-cyano-1-(2-fluoro-benzyl)-4-hydroxy-1H-pyrrolo[2,3-c]pyridine-5-carbonyl]-amino}-acetic acid (G), {[7-cyano-4-hydroxy-1-(3-methoxy-benzyl)-1H-pyrrolo[2,3-c]pyridine-5-carbonyl]-amino}-acetic acid (E), [(7-cyano-4-hydroxy-1-naphthalen-2-ylmethyl-1H-pyrrolo[2,3-c]pyridine-5-carbonyl)-amino]-acetic acid (F).

Suitable compounds for use in the methods and medicaments of the invention may be identified using any conventionally known methods. Suitable assay methods are well known in the art. For example, compounds may be tested for their ability to inhibit the activity of a HIF prolyl hydroxylase in an enzyme assay as described in Example 1. Compounds are combined with radiolabeled a-ketoglutarate, a hydroxylatable HIFα peptide, and a HIF prolyl hydroxylase, e.g., EGLN3 under conditions where, in the absence of compound, the HIF prolyl hydroxylase is capable of hydroxylating the HIFα peptide and converting the α-ketoglutarate to succinate and carbon dioxide; and levels of liberated carbon dioxide are measured, wherein a reduction in the amount of liberated carbon dioxide in the presence of compound identifies an inhibitor of HIF prolyl hydroxylase. Alternatively, compounds may be tested for their ability to inhibit the activity of a HIF prolyl hydroxylase indirectly using the HIFα stabilization assay of Example 2.

In certain aspects, the methods of the present invention further comprise treatment of the subject with a second therapeutic compound selected from the group consisting of ACEI, ARBs, α-blockers, BBs, vasodilators, CCBs, and statins.

### Pharmaceutical Formulations and Routes of Administration

The PHIs used in the methods of the present invention can be administered directly to the subject or in medicaments (pharmaceutical formulations) containing excipients, as is well known in the art. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.) Present methods of treatment can comprise administration of an effective amount of a compound or medicament to a subject having or at risk for having high blood pressure or hypertension. In some embodiments, the subject is a mammalian subject, and in particular embodiments, the subject is a human subject.

In certain aspects, the PHI may be administered or formulated together with a second therapeutic compound. In particular embodiments, the methods and medicaments of the invention further comprise administering or formulating the PHI in combination with at least one therapeutic agent selected from the group consisting of ACE inhibitors, ARBs, a-blockers, BBs, vasodilators, diuretics, CCBs, and statins.

An effective amount, e.g., dose, of compound can readily be determined by routine experimentation, as can an effective and convenient route of administration and an appropriate formulation.

Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.) Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracistemal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

The effective amount or therapeutically effective amount is the amount of the agent or pharmaceutical composition that will elicit the biological or medical response of a tissue, system, animal, or human that is being sought by the researcher, veterinarian, medical doctor, or other clinician, e.g., reduction in blood pressure, etc. A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Compounds that exhibit high therapeutic indices are preferred. Dosages preferably fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, e.g., suitable reduction or maintenance of blood pressure, etc, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### HIF Prolyl Hydroxylase Inhibition Assay

Ketoglutaric acid α-[1-14C]-sodium salt, alpha-ketoglutaric acid sodium salt, and HPLC purified peptide may be obtained from commercial sources, e.g., Perkin-Elmer (Wellesley Mass.), Sigma-Aldrich, and SynPep Corp. (Dublin Calif.), respectively. Peptides for use in the assay may be fragments of HIFα including, but not limited to, any fragment retaining at least one functional or structural characteristic of HIFα. Fragments of HIFα include, e.g., the regions defined by human HIF-1α from amino acids 401 to 603 (Huang et al., supra), amino acid 531 to 575 (Jiang et al. (1997) J. Biol. Chem. 272:19253-19260), amino acid 556 to 575 (Tanimoto et al., supra), amino acid 557 to 571 (Srinivas et al. (1999) Biochem. Biophys. Res. Commun. 260:557-561), and amino acid 556 to 575 (Ivan and Kaelin (2001) Science 292:464-468). Further, HIFα fragments include any fragment containing at least one occurrence of the motif LXXLAP, e.g., such as occurs in the human HIF-1α native sequence from L₃₉₇ to P₄₀₂, and from L₅₅₉ to P₅₆₄. Additional fragments that may be used in the assay are fragments of HIFα disclosed in International Publication WO 2005/118836, incorporated by reference herein. For example, a HIF peptide for use in the screening assay may comprise [methoxycoumarin]-DLDLEALAPYIPADDDFQL-amide.

HIF-PH, e.g., HIF-PH2 (EGLN1), can be expressed in, e.g., insect Hi5 cells, and partially purified, e.g., through a SP ion exchange chromatography column. Enzyme activity is determined by capturing ¹⁴CO₂ using an assay described by Kivirikko and Myllyla (1982, Methods Enzymol. 82:245-304). Assay reactions contain 50 mM HEPES (pH 7.4), 100 μM α-ketoglutaric acid sodium salt, 0.30 μCi/mL ketoglutaric acid□ α-[1-¹⁴C]-sodium salt, 40 μM FeSO4, 1 mM ascorbate, 1541.8 units/mL Catalase, with or without 50 μM peptide substrate and various concentrations of compound of the invention. Reactions are initiated by addition of HIF-PH enzyme.

The peptide-dependent percent turnover is calculated by subtracting percent turnover in the absence of peptide from percent turnover in the presence of substrate peptide. Percent inhibition and IC₅₀ are calculated using peptide-dependent percent turnover at given inhibitor concentrations. Calculation of IC50 values for each inhibitor is conducted using GraFit software (Erithacus Software Ltd., Surrey UK). The IC₅₀ for exemplified compounds in the EGLN3 assay ranged from approximately 6-1160 nanomolar. Thus, compounds used in the methods and medicaments as exemplified below are inhibitors of HIF prolyl hydroxylase.

### Example 2

### Cell-Based HIFα Stabilization Assay

Human cells derived from various tissues are separately seeded into 35 mm culture dishes, and grown at 37° C., 20% O₂, 5% CO₂ in standard culture medium, e.g., DMEM (Dulbecco's modification of Eagle's medium), 10% FBS (fetal bovine serum). When cell layers reach confluence, the media is replaced with OPTI-MEM media (Invitrogen Life Technologies, Carlsbad Calif.), and cell layers are incubated for approximately 24 hours in 20% O₂, 5% CO₂ at 37° C. Compound or 0.013% DMSO (dimethyl sulfoxide) is then added to existing medium and incubation is continued overnight.

Following incubation, the media is removed, centrifuged, and stored for analysis (see VEGF and EPO assays below). The cells are washed two times in cold phosphate buffered saline (PBS) and then lysed in 1 mL of 10 mM Tris (pH 7.4), 1 mM EDTA, 150 mM NaCl, 0.5% IGEPAL (Sigma-Aldrich, St. Louis Mo.), and a protease inhibitor mix (Roche Molecular Biochemicals) for 15 minutes on ice. Cell lysates are centrifuged at 3,000×g for 5 minutes at 4° C., and the cytosolic fractions (supernatant) are collected. The nuclei (pellet) are resuspended and lysed in 100 μL of 20 mM HEPES (pH 7.2), 400 mM NaCl, 1 mM EDTA, 1 mM dithiothreitol, and a protease mix (Roche Molecular Biochemicals), centrifuged at 13,000×g for 5 minutes at 4° C., and the nuclear protein fractions (supernatant) are collected.

Nuclear fractions are analyzed for HIF-1α using a QUANTIKINE immunoassay (R&D Systems, Inc., Minneapolis Minn.) according to the manufacturer's instructions.

### Example 3

### Treatment Using a HIF Prolyl Hydroxylase Inhibitor Reduces Hypertension in an Animal Model of Chronic Kidney Disease

A characteristic pathophysiology found in the uremic chronic kidney disease (CKD) population is hypertension. The rat remnant kidney model generated by 5/6 nephrectomy is a well-established animal model of CKD that exhibits this pathology. (Priyadarshi et al. (2002) Kidney Int 61:542-546; Coleman et al. (2006) Proc Natl Acad Sci USA 103:5965-5970.) The surgical procedure used to generate the rat remnant kidney was performed according to modification of the procedure described by Priyadarshi et al. (supra) or according to modification of the procedure described by Tanaka et al. (2005, Lab Invest 85:1292-1307). Separate studies used either male Sprague Dawley rats (280-300 g) or female Wistar rats (220-250 g).

Animals were divided into two groups of which one group (sham) underwent sham surgery and the other group (Nx) was subjected to 5/6 nephrectomy as follows. Animals were anesthetized under isoflurane, and a midline abdominal incision was made, followed by blunt dissection of the renal pedicles. Two or three branches of the left renal artery were ligated to infarct two thirds of the left kidney, while the right kidney underwent simultaneous nephrectomy. The infarct was verified by observing the change in kidney color. Animal body temperature was maintained constant, and warm (˜37° C.) saline (1.0 mL) was administered directly into the abdomen. Thereafter, the incision was sutured and the animal was allowed to recover and have free access to regular food and water. Animal body weight and mortality were closely monitored.

Starting 35 days after surgery, Nx and sham animals were randomly divided into groups and treated with either vehicle or compound A. Compound was dosed three times per week by oral gavage at 20 or 40 mg/kg compound per dose. Blood pressure (BP) was measured using a Kent XBP1000 system (Kent Scientific Corp., Torrington, Conn.) or a Muromachi tail-cuff blood pressure system (Muromachi Kikai Co., Ltd., Tokyo, Japan), both having volume/pressure recording technology and used according to the manufacturer's instruction. Rats were slightly warmed under a lamp and conditioned carefully before the measurement. BP was recorded as the mean value of 3 separate measurements obtained at each session and reported herein as mean±SEM. Results are compared using 1-way analysis of variance (ANOVA) and Student-Newman-Keuls method (SigmaStat, SPSS Science, Chicago, Ill.).

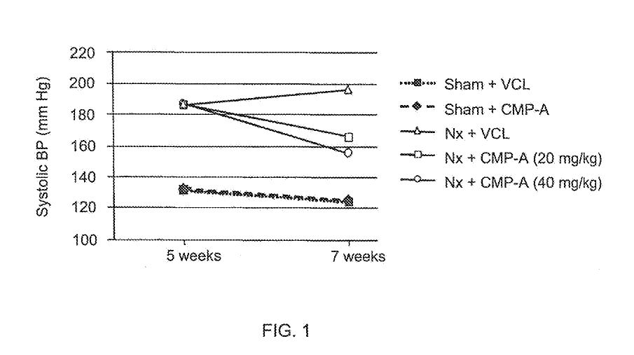

Treatment of Nx animals with compound according to the methods of the present invention consistently results in lower blood pressure relative to treatment with vehicle alone. For example, in one experiment, treatment of Nx rats with compound A at 20 or 40 mg/kg reversed pre-existing hypertension. (FIG. 1.) Baseline measurements of systolic blood pressure (SBP) taken 3 weeks after surgery indicated that hypertension had developed to the same degree (approximately a 42% increase in SBP on average) in all Nx groups compared to sham groups. SBP was significantly reduced (p<0.05) in Nx rats treated with compound A at 20 mg/kg (164.7±26.7 mmHg) and at 40 mg/kg (155.4±42.9 mmHg) compared to vehicle-treated Nx rats (195.7±24.4).

These results showed that methods and medicaments of the present invention were effective at reducing systolic blood pressure in a rat model of hypertensive chronic kidney disease. Additionally, these results showed that methods and medicaments of the present invention are effective at reducing blood pressure in subjects with CKD. These results further demonstrated that methods and medicaments of the present invention are effective for treating or preventing hypertension.

### Example 4

### Treatment Using a Variety of HIF Prolyl Hydroxylase Inhibitors Reduces Hypertension in an Animal Model System

In another series of experiments, rats subjected to 5/6 nephrectomy were treated 35 days after surgery according to the dosing schedule described in Example 3 above with a HIF prolyl hydroxylase inhibitor. Individual compound selected from the group consisting of compounds A and C-K were administered by oral gavage at 8, 20, 30, or 40 mg/kg (see Table 1 and Table 2). Systolic blood pressure was recorded at day 35 (baseline) and again immediately following the last dose of compound. Table 1 and Table 2 show changes in systolic blood pressure following four weeks of oral administration of various compounds of the present invention to Nx rats. Systolic blood pressure is reported in Table 1 as change from baseline (the extent that compound reduced blood pressure from baseline levels) and Table 2 as change from vehicle (the extent that compound reduced blood pressure as compared to vehicle treated Nx rats).

As shown below in Table 1, Nx rats treated using the methods or medicaments of the present invention showed a reduction in systolic blood pressure from baseline, indicating that the methods and medicaments reduce blood pressure in hypertensive animals.

TABLE 1

| | | SBP (mmHg) |
|---|---|---|
| Compound | Dose (mg/kg) | Change from Baseline |
| A | 40 | −31.10 |
| C | 30 | −13.72 |
| D | 8 | −22.14 |
| D | 20 | −9.25 |
| E | 20 | −7.12 |
| F | 8 | −10.58 |

As shown below in Table 2, Nx rats treated using the methods or medicaments of the present invention showed a prevention in increased systolic blood pressure over time relative to vehicle-treated animals, indicating that the methods and medicaments prevent progression in hypertension in animals with kidney disease.

TABLE 2

| | | SBP (mmHg) |
|---|---|---|
| Compound | Dose (mg/kg) | Change from Vehicle |
| A | 40 | −40.31 |
| C | 30 | −34.03 |
| G | 8 | −19.27 |
| H | 20 | −15.73 |
| I | 20 | −11.00 |
| D | 8 | −12.4 |
| J | 8 | −2.66 |
| D | 20 | −24.73 |
| E | 20 | −18.55 |
| F | 8 | −23.88 |
| K | 8 | −19.92 |

These results showed that methods and medicaments of the present invention were effective at reducing elevated blood pressure associated with kidney disease, specifically, systolic blood pressure in a rat model of hypertensive chronic kidney disease. Additionally, these results showed that methods and medicaments of the present invention are effective at reducing blood pressure in subjects with CKD. These results further demonstrated that methods and medicaments of the present invention are effective for treating or preventing hypertension.

### Example 5

### Methods and Medicaments were Therapeutically Effective in Reducing Blood Pressure in Human Subjects with CKD

Hypertension is a common condition in patients with chronic kidney disease. (Agarwal et al. (2005) Hypertension 46:514-520.) The effect of a compound of the present invention on blood pressure in anemic and non-anemic human subjects with advanced stage chronic kidney disease was examined. All study subjects had chronic kidney disease (CKD), defined as having a glomerular filtration rate (GFR)<59 ml/min. Some of these CKD subjects also had anemia, defined as having hemoglobin (Hb) levels<11 g/dL. Subjects were orally administered compound A or a placebo two or three times per week for four weeks. Blood pressure readings (i.e. systolic, diastolic, and mean arterial pressure) were taken at various times with a sphygmomanometer according to standard assessments.

In one series of experiments, the effect of methods and medicaments of the present invention on blood pressure in anemic and non-anemic subjects with advanced stage chronic kidney disease was examined over a 24 hour period. Subjects were orally administered compound A or a placebo and blood pressure readings were taken at 1, 2, 3, 4, 6, 12, and 24 hours following treatment. All subjects showed small increases in blood pressure at 1 and 2 hours post dosing, regardless of whether compound A or placebo was administered (data not shown).

Table 3, Table 4, and Table 5 below show hourly changes in mean arterial pressure (MAP), systolic blood pressure (SBP), and diastolic blood pressure (DBP) following oral administration of compound X to human CKD subjects. As shown below in Table 3, CKD subjects (anemic and non-anemic) administered compound A showed a mean reduction in mean arterial pressure from baseline levels at all timepoints. Similarly, CKD subjects treated with compound A showed a mean reduction in both systolic and diastolic blood pressure (see Table 4 and Table 5). In contrast, subjects administered placebo generally showed a mean elevation in blood pressure (i.e., MAP, SBP, and DBP) from baseline levels.

TABLE 3

| | Mean | Mean change from Baseline | | | | |
|---|---|---|---|---|---|---|
| | Baseline | MAP (mmHg) | | | | |
| Treatment | MAP | 3 | 4 | 6 | 12 | 24 |
| Group | (mmHg) | hour | hour | hour | hour | hour |
| Cmpd A | 91.7 | −4.35 | −6.60 | −9.18 | −3.29 | −2.55 |
| (All Subjects) | | | | | | |
| Placebo | 93.6 | 5.40 | 4.35 | 1.27 | 1.72 | 3.55 |
| (All Subjects) | | | | | | |
| Cmpd A | 94.1 | −11.10 | −14.67 | −12.33 | −2.00 | −3.77 |
| (Anemic) | | | | | | |
| Placebo | 97.5 | −2.77 | −1.20 | −4.23 | −3.67 | 0.33 |
| (Anemic) | | | | | | |
| Cmpd A | 87.3 | −3.81 | −3.58 | −8.00 | −3.78 | −8.00 |
| (Non-Anemic) | | | | | | |
| Placebo | 87.8 | 13.57 | 9.90 | 6.77 | 7.10 | 6.77 |
| (Non-Anemic) | | | | | | |

TABLE 4

| | Mean | Mean change from Baseline Systolic | | | | |
|---|---|---|---|---|---|---|
| | Baseline | BP (mmHg) | | | | |
| Treatment | Systolic BP | 3 | 4 | 6 | 12 | 24 |
| Group | (mmHg) | hour | hour | hour | hour | hour |
| Cmpd A | 137.1 | −2.09 | −7.00 | −13.45 | −4.73 | −4.45 |
| (All Subjects) | | | | | | |
| Placebo | 137.4 | 2.67 | 1.83 | −2.00 | −2.33 | 5.50 |
| (All Subjects) | | | | | | |
| Cmpd A | 138.7 | −6.33 | −19.00 | −17.33 | 3.67 | −5.00 |
| (Anemic) | | | | | | |
| Placebo | 140.7 | −6.00 | −7.33 | −11.00 | −14.67 | −0.67 |
| (Anemic) | | | | | | |
| Cmpd A | 134.0 | −0.50 | −2.50 | −12.00 | −7.88 | −4.25 |
| (Non-Anemic) | | | | | | |
| Placebo | 132.5 | 11.33 | 11.00 | 7.00 | 10.00 | 11.67 |
| (Non-Anemic) | | | | | | |

TABLE 5

| | Mean | Mean change from Baseline Diastolic | | | | |
|---|---|---|---|---|---|---|
| | Baseline | BP (mmHg) | | | | |
| Treatment | Diastolic BP | 3 | 4 | 6 | 12 | 24 |
| Group | (mmHg) | hour | hour | hour | hour | hour |
| Cmpd A | 69.0 | −5.45 | −6.36 | −7.00 | −2.55 | −1.55 |
| (All Subjects) | | | | | | |
| Placebo | 71.8 | 6.67 | 5.50 | 2.83 | 3.67 | 2.50 |
| (All Subjects) | | | | | | |
| Cmpd A | 71.8 | −13.67 | −12.67 | −10.00 | −5.00 | −3.33 |
| (Anemic) | | | | | | |
| Placebo | 75.8 | −1.00 | 2.00 | −0.67 | 2.00 | 1.00 |
| (Anemic) | | | | | | |
| Cmpd A | 63.8 | −2.38 | −4.00 | −5.88 | −1.63 | −0.88 |
| (Non-Anemic) | | | | | | |
| Placebo | 65.8 | 14.33 | 9.00 | 6.33 | 5.33 | 4.00 |
| (Non-Anemic) | | | | | | |

These results showed that the methods and medicaments of the present invention were effective at reducing mean arterial pressure, systolic blood pressure, and diastolic blood pressure in human subjects with chronic kidney disease. Additionally, these results showed that the methods and medicaments of the present invention are effective at reducing blood pressure in both anemic and non-anemic human subjects with chronic kidney disease. Hypertension is a common condition in patients with CKD. These results demonstrated that the methods and medicaments of the present invention are effective at reducing blood pressure in these patients; therefore, these results showed that the methods and medicaments of the present invention are effective at treating hypertension in a subject. CKD subjects treated with placebo showed an elevation in mean arterial pressure, systolic blood pressure, and diastolic blood pressure from baseline, however, this elevation was prevented in CKD subjects treated using the methods and medicaments of the present invention. Thus, the methods and medicaments of the present invention are useful for preventing elevations in mean arterial pressure, systolic blood pressure, and diastolic blood pressure in CKD subjects. These results further suggested that the methods and medicaments of the present invention would be useful for preventing hypertension in subjects with CKD

### Example 6

### Methods and Medicaments were Therapeutically Effective in Treating Hypertension in Human Subjects with CKD

In another series of experiments, the effects of a medicament of the present invention on blood pressure in anemic and non-anemic subjects with chronic kidney disease were determined over a four week period. All subjects were orally administered compound A or a placebo two or three-times per week for four weeks and blood pressure readings were taken weekly.

Table 6, Table 7, and Table 8 below show weekly changes in mean arterial pressure (MAP), systolic blood pressure (SBP), and diastolic blood pressure (DBP) following oral administration of compound A to human CKD subjects. As shown below in Table 6, CKD subjects (anemic and non-anemic) administered compound A showed a mean reduction in mean arterial pressure from baseline levels at all timepoints. Similarly, CKD subjects treated with compound A showed a mean reduction in both systolic and diastolic blood pressure (see Table 7 and Table 8). In contrast, subjects administered placebo generally showed a mean elevation in blood pressure (i.e., MAP, SBP, and DBP) from baseline levels.

TABLE 6

| | | Mean change from | | | |
|---|---|---|---|---|---|
| | Mean Baseline | Baseline MAP (mmHg) | | | |
| Treatment Group | MAP (mmHg) | Week 1 | Week 2 | Week 3 | Week 4 |
| Cmpd A | 91.7 | −4.28 | −5.44 | −4.03 | −2.85 |
| (All Subjects) | | | | | |
| Placebo | 93.6 | 3.93 | 4.86 | 0.04 | 5.70 |
| (All Subjects) | | | | | |
| Cmpd A | 94.1 | −4.57 | −6.13 | −5.47 | −3.08 |
| (Anemic) | | | | | |
| Placebo | 97.5 | 2.54 | 4.60 | −0.32 | 9.20 |
| (Anemic) | | | | | |
| Cmpd A | 87.3 | −3.67 | −3.95 | −1.14 | −2.48 |
| (Non-Anemic) | | | | | |
| Placebo | 87.8 | 5.68 | 5.18 | 0.50 | 1.33 |
| (Non-Anemic) | | | | | |

TABLE 7

| | Mean Baseline | Mean change from Baseline | | | |
|---|---|---|---|---|---|
| Treatment | Systolic BP | Systolic BP (mmHg) | | | |
| Group | (mmHg) | Week 1 | Week 2 | Week 3 | Week 4 |
| Cmpd A | 137.1 | −5.45 | −9.47 | −8.97 | −5.54 |
| (All Subjects) | | | | | |
| Placebo | 137.4 | 5.89 | 6.22 | −3.11 | 6.44 |
| (All Subjects) | | | | | |
| Cmpd A | 138.7 | −4.80 | −10.77 | −11.40 | −7.25 |
| (Anemic) | | | | | |
| Placebo | 140.7 | 4.80 | 4.20 | −7.00 | 11.80 |
| (Anemic) | | | | | |
| Cmpd A | 134.0 | −6.86 | −6.67 | −4.10 | −2.80 |
| (Non-Anemic) | | | | | |
| Placebo | 132.5 | 7.25 | 8.25 | 1.75 | −0.25 |
| (Non-Anemic) | | | | | |

TABLE 8

| | Mean Baseline | Mean change from Baseline | | | |
|---|---|---|---|---|---|
| Treatment | Diastolic | Diastolic BP (mmHg) | | | |
| Group | BP (mmHg) | Week 1 | Week 2 | Week 3 | Week 4 |
| Cmpd A | 69.0 | −3.66 | −3.37 | −1.53 | −1.54 |
| (All Subjects) | | | | | |
| Placebo | 71.8 | 2.78 | 4.00 | 1.44 | 5.17 |
| (All Subjects) | | | | | |
| Cmpd A | 71.8 | −4.43 | −3.77 | −2.50 | −1.13 |
| (Anemic) | | | | | |
| Placebo | 75.8 | 1.40 | 4.80 | 3.00 | 7.90 |
| (Anemic) | | | | | |
| CmpdA | 63.8 | −2.00 | −2.50 | 0.40 | −2.20 |
| (Non-Anemic) | | | | | |
| Placebo | 65.8 | 4.50 | 3.00 | −0.50 | 1.75 |
| (Non-Anemic) | | | | | |

These results showed that the methods and medicaments of the present invention were effective at reducing mean arterial pressure, systolic blood pressure, and diastolic blood pressure in human subjects with chronic kidney disease. Additionally, these results showed that the methods and medicaments of the present invention are effective at reducing blood pressure in both anemic and non-anemic human subjects with chronic kidney disease. Hypertension is a common condition in patients with CKD. These results demonstrated that the methods and medicaments of the present invention are effective at reducing blood pressure in these patients; therefore, these results showed that the methods and medicaments of the present invention are effective at treating hypertension in a subject. CKD subjects treated with placebo generally showed an elevation in mean arterial pressure, systolic blood pressure, and diastolic blood pressure from baseline. However, this elevation was prevented in CKD subjects treated using the methods and medicaments of the present invention. Thus, the methods and medicaments of the present invention are useful for preventing elevations in mean arterial pressure, systolic blood pressure, and diastolic blood pressure in CKD subjects. These results further suggested that the methods and medicaments of the present invention would be useful for preventing hypertension in subjects with CKD.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for treating hypertension in a subject having hypertension and kidney disease, the method comprising administering to the subject an effective amount of a compound that inhibits the activity of a hypoxia-inducible factor (HIF) prolyl hydroxylase enzyme, thereby treating hypertension in the subject.

2. The method of claim 1, wherein the subject has anemia.

3. The method of claim 1, wherein the subject does not have anemia.

4. The method of claim 1, wherein the compound is a structural mimetic of 2-oxoglutarate that inhibits HIF prolyl hydroxylase enzyme competitively with respect to 2-oxoglutarate and noncompetitively with respect to iron.

5. The method of claim 3, wherein the compound is a compound of formula A: 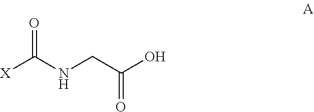
wherein X is an optionally substituted heterocyclic moiety.

6. The method of claim 4, wherein the compound is a compound of formula I: 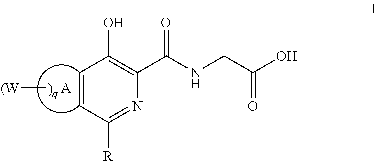
wherein
A is selected from a benzene or pyrrole ring;
q is 1, 2 or 3;
W is selected from (C₁-C₃)-alkyl, (C₁-C₃)-alkoxy or (C₆-C₁₀)-aryloxy, each of which may be unsubstituted or substituted by one or more halo, (C₁-C₃)-alkyl, (C₁-C₃)-alkoxy, or (C₆-C₁₀)-aryl; and
R is selected from hydrogen, alkyl, or cyano;
or a pharmaceutically acceptable salt, ester, or prodrug thereof.

7. The method of claim 1, wherein the subject is a mammalian subject.

8. The method of claim 1, wherein the subject is a human subject.

9. The method of claim 1, wherein the hypertension is selected from the group consisting of mild hypertension, moderate hypertension, severe hypertension, and very severe hypertension.

10. A method for treating hypertension in a subject having hypertension, the method comprising administering to the subject an effective amount of a compound of formula I: 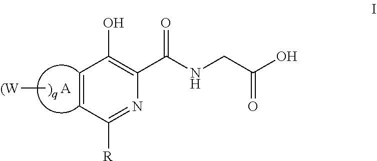
wherein
A is selected from a benzene or pyrrole ring;
q is 1, 2 or 3;
W is selected from (C₁-C₃)-alkyl, (C₁-C₃)-alkoxy or (C₆-C₁₀)-aryloxy, each of which may be unsubstituted or substituted by one or more halo, (C₁-C₃)-alkyl, (C₁-C₃)-alkoxy, or (C₆-C₁₀)-aryl; and
R is selected from hydrogen, alkyl, or cyano;
or a pharmaceutically acceptable salt, ester, or prodrug thereof.

11. The method of claim 10, wherein the compound is selected from the group consisting of a compound of formula I(a) and a compound of formula I(b): 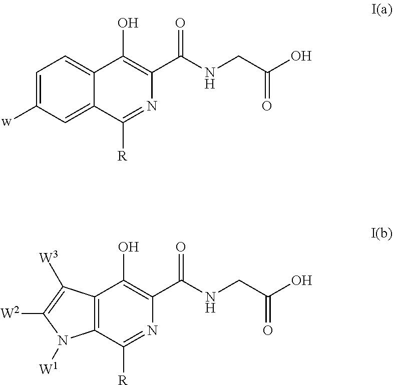
wherein
W is selected from (C₁-C₃)-alkoxy or (C₆-C₁₀)-aryloxy, each of which may be unsubstituted or substituted by a substituent selected from the group consisting of (C₁-C₃)-alkyl and (C₁-C₃)-alkoxy;
W¹ is selected from (C₁-C₃)-alkyl, which may be unsubstituted or substituted by one or more (C₁-C₃)-alkoxy or (C₆-C₁₀)-aryl;
W² and W³ are each independently selected from halo or (C₁-C₃)-alkyl; and
R is selected from hydrogen, alkyl, or cyano;
or a pharmaceutically acceptable salt, ester, or prodrug thereof.

12. The method of claim 10, wherein the compound is selected from the group consisting of {[4-hydroxy-7-(4-methoxy-phenoxy)-isoquinoline-3-carbonyl]-amino}-acetic acid, [(4-hydroxy-1-methyl-7-phenoxy-isoquinoline-3-carbonyl)-amino]-acetic acid, [(1-cyano-4-hydroxy-5-phenoxyisoquinoline-3-carbonyl)-amino]-acetic acid, [(1-cyano-4-hydroxy-7-isopropoxy-isoquinoline-3-carbonyl)-amino]-acetic acid, {[3-bromo-7-cyano-4-hydroxy-1-(4-methoxy-benzyl)-1H-pyrrolo[2,3-c]pyridine-5-carbonyl]-amino}-acetic acid, {[3-chloro-7-cyano-4-hydroxy-1-(4-methoxy-benzyl)-1H-pyrrolo[2,3-c]pyridine-5-carbonyl]-amino}-acetic acid, {[2,3-dichloro-7-cyano-4-hydroxy-1-(4-methoxy-benzyl)-1Hpyrrolo[2,3-c]pyridine-5-carbonyl]-amino}-acetic acid, {[1-cyano-7-(2,6-dimethyl-phenoxy)-4-hydroxyisoquinoline-3-carbonyl]-amino}-acetic acid, {[7-cyano-1-(2-fluoro-benzyl)-4-hydroxy-1H-pyrrolo[2,3-c]pyridine-5-carbonyl]-amino}-acetic acid, {[7-cyano-4-hydroxy-1-(3-methoxy-benzyl)-1H-pyrrolo[2,3-c]pyridine-5-carbonyl]-amino}-acetic acid, [(7-cyano-4-hydroxy-1-naphthalen-2-ylmethyl-1H-pyrrolo[2,3-c]pyridine-5-carbonyl)-amino]-acetic acid.

13. The method of claim 10, wherein the subject is a mammalian subject.

14. The method of claim 10, wherein the subject is a human subject.

15. The method of claim 10, wherein the hypertension is selected from the group consisting of mild hypertension, moderate hypertension, severe hypertension, and very severe hypertension.
